(19) United States
(12) Plant Patent Application Publication
(10) Pub. No.: US 2014/0053305 P1
(43) Pub. Date: Feb. 20, 2014

(54) Campanula plant named 'PKMpos01'

(50) ;

(71) Applicant: Christian Hald Madsen, Korsor (DK)

(72) Inventor: Christian Hald Madsen, Korsor (DK)

(73) Assignee: Gartneriet PKM A/S

(21) Appl. No.: 13/573,056

(22) Filed: Aug. 17, 2012

(51) Int. Cl. A01H 5/00

(52) U.S. Cl. PLT/414

(57) ABSTRACT

A distinct cultivar of Campanula plant named ‘PKMpos01’ characterized by its compact plant habit; globular plant form; vigorous growth habit; upright violet-blue flowers and large leaves.

### LATIN NAME OF GENUS AND SPECIES OF THE PLANT CLAIMED

Campanula poschskaskyana

### VARIETY DENOMINATION

‘PKMPOS01’

### BACKGROUND OF THE INVENTION

The present invention relates to a new and distinct cultivar of Campanula plant, botanically known as Campanula poschskaskyana, hereinafter referred to by the name ‘PKMPOS01’.

The new Campanula, ‘PKMPOS01’, is a product of a planned breeding program conducted by the inventor, Christian Hald Madsen, in Søhus, Denmark. The new Campanula originated from a cross made by the inventor in June 2009 between a proprietary selection of Campanula poschskaskyana designated 37.09.1004 (breeder reference, unpatented) as the female parent and a proprietary selection of Campanula poschskaskyana designated 37 (breeder reference, unpatented) as the male parent. The new Campanula ‘PKMPOS01’ was discovered and selected by the inventor in June 2010 within the progeny of the stated cross in a controlled environment in Søhus, Denmark. The inventor selected ‘PKMPOS01’ on the basis of its compact and freely flowering plant habit.

Asexual reproduction of the new Campanula cultivar by terminal cuttings has demonstrated that the combination of characteristics as herein disclosed for the new cultivar are firmly fixed and retained through successive generations of asexual reproduction. The new cultivar reproduces true-to-type.

### SUMMARY OF THE INVENTION

The following traits have been repeatedly observed and are determined to be the unique characteristics of ‘PKMPOS01’. These characteristics in combination distinguish ‘PKMPOS01’ as a new and distinct cultivar:

1. Upright plant habit with long upright stems.

2. Bushy globular plant form.

3. Vigorous growth habit, but still needing less chemical growth retardation.

4. Small violet-blue flowers with wide and short flowers.

5. Large quantity of buds.

6. No need for vernalization

Plants of the cultivar ‘PKMPOS01’ can be compared to the Campanula portenschlagiana Schult. cultivar ‘PKMP05’ (patented, U.S. Plant Pat. No. 17,188). In side-by-side comparisons conducted by the inventor in Stige, Denmark, plants of the instant cultivar ‘PKMPOS01’ and plants of the cultivar ‘PKMP05’ differ in the following characteristics:

1. Plants of ‘PKMPOS01’ have longer stems than plants of ‘PKMP05’.

2. Plants of ‘PKMPOS01’ have a longer leaf length than plants of ‘PKMP05’.

3. Plants of ‘PKMPOS01’ have violet-blue petals whereas plants of ‘PKMP05’ have purple petals.

Plants of the cultivar ‘PKMPOS01’ can also be compared to the parent Campanula poschskaskyana cultivars 37.09.1004 (female parent) and 37 (male parent) (breeder references, unpatented). Plants of the instant cultivar ‘PKMPOS01’ and plants of the parent cultivars 37.09.1004 and 37 differ in the following characteristics:

1. Plants of ‘PKMPOS01’ are more upright than plants of the parental cultivars.

2. Plants of ‘PKMPOS01’ are more freely flowering than plants of the parental cultivars.

### BRIEF DESCRIPTION OF THE PHOTOGRAPHS

The accompanying colored photographs illustrate the overall appearance of the new Campanula ‘PKMPOS01’, showing the colors as true as it is reasonably possible to obtain in colored reproductions of this type. Colors in the photographs may differ slightly from the color values cited in the detailed botanical description, which more accurately describe the actual colors of ‘PKMPOS01’.

FIG. 1: The photograph comprises a side perspective view of a typical flowering plant of PKMpos01.

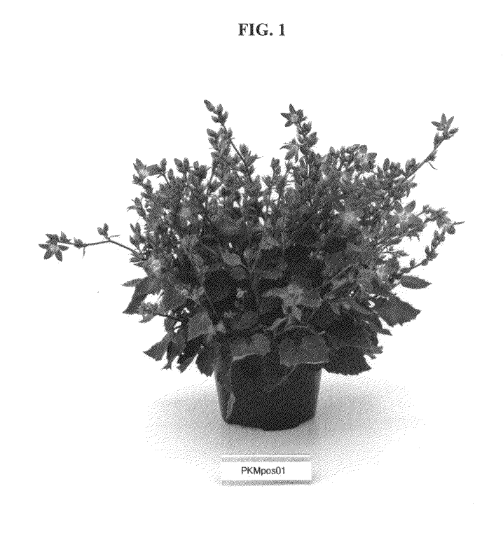

FIG. 2: The photograph comprises a close-up view of a typical flower bud of PKMpos01.

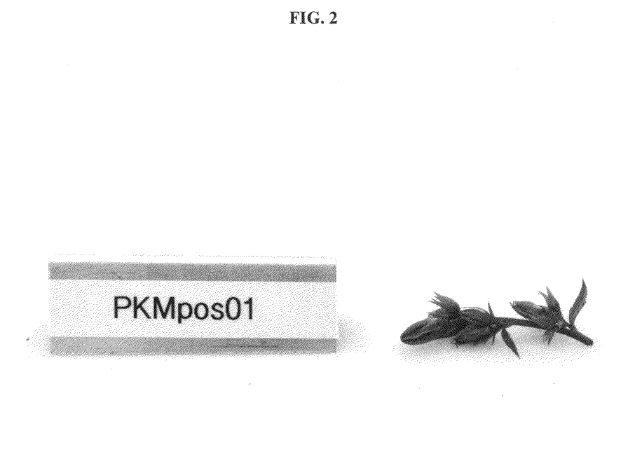

FIG. 3: The photograph comprises a close-up side perspective view of a typical flower of PKMpos01.

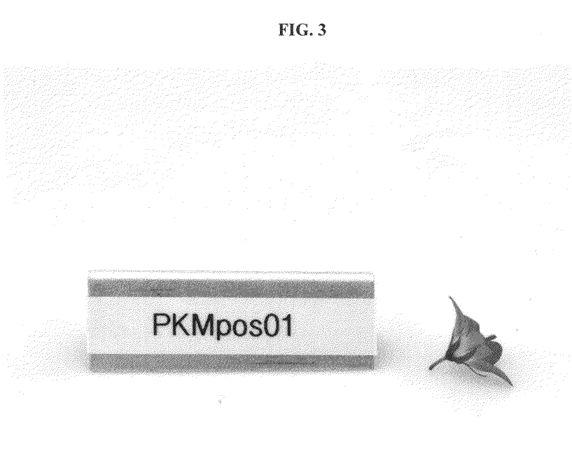

FIG. 4: The photograph comprises a close-up view of a typical flower of PKMpos01.

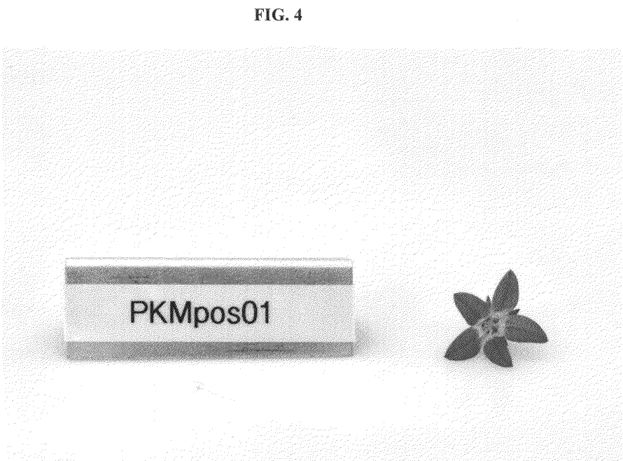

FIG. 5: The photograph comprises a close-up view of a typical apical leaf of PKMpos01.

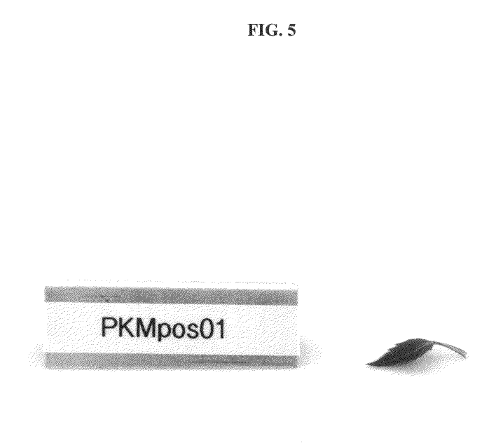

FIG. 6: The photograph comprises a close-up view of a typical basal leaf of PKMpos01.

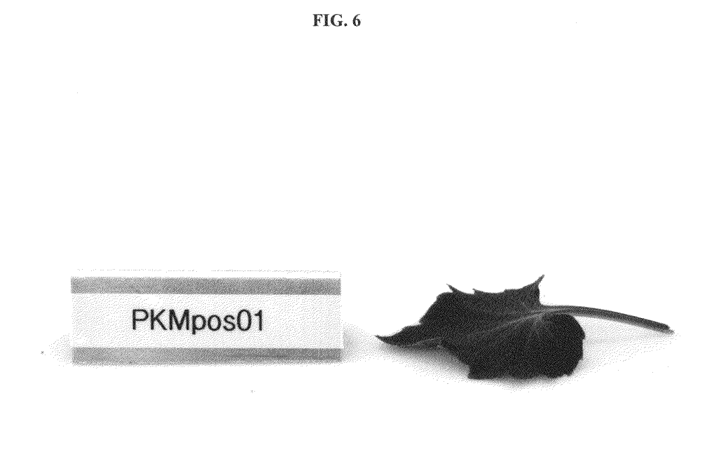

FIG. 7: The photograph comprises a comparison view of a typical flowering plant of PKMpos01 with a typical flowering plant of PKMp05.

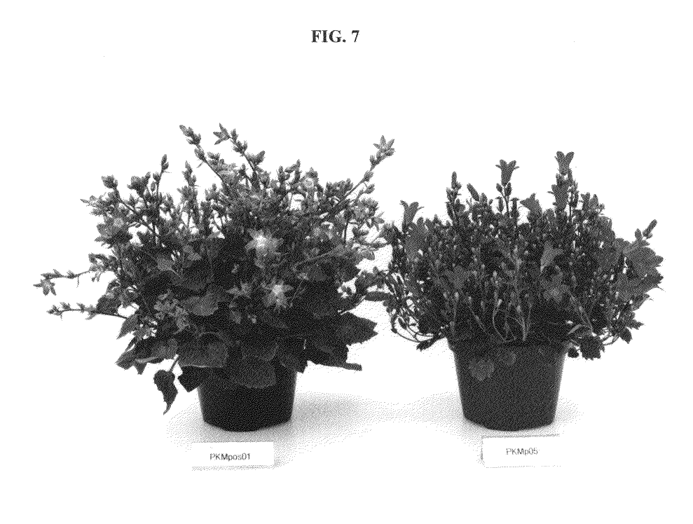

FIG. 8: The photograph comprises a comparison view of a typical flower of PKMpos01 with a typical flower of PKMp05.

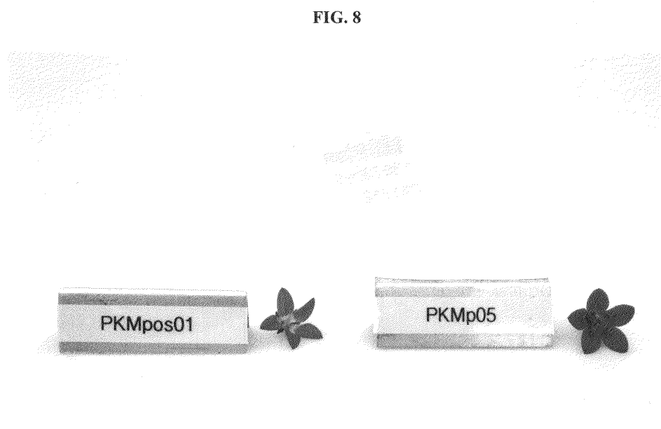

### DETAILED BOTANICAL DESCRIPTION

The new Campanula ‘PKMPOS01’ has not been observed under all possible environmental conditions. The phenotype of the new cultivar may vary with variations in environment such as temperature, light intensity, day length, and fertility level without any variance in genotype.

The aforementioned photographs, together with the following observations, measurements and values describe plants of the new Campanula ‘PKMPOS01’ as grown under greenhouse conditions. No growth retardants were used.

The age of the ‘PKMPOS01’ plants described is about 14 weeks after cutting. Color references are made to The Royal Horticultural Society Colour Chart (R.H.S.), 5^{th} edition, except where general colors of ordinary significance are used.
Classification:
   Botanical.—Campanula poschskaskyana.
Parentage:
   Female or seed parent.—Campanula poschskaskyana designated 37.09.1004 (breeder reference, unpatented).
   Male or pollen parent.—Campanula poschskaskyana designated 37 (breeder reference, unpatented).
Propagation:
   Type cutting.—Terminal vegetative cuttings.
   Time to initiate roots.—About 10 to 14 days at 18 to 21° C. in tunnels in a greenhouse.
   Root description.—Fine, well branched.
Plant description:
   Form.—Perennial, rosette plant with upright plant habit. Campanulate flowers in racemes. Freely branching with lateral branches forming at every node; dense and bushy.
   Crop time.—After rooting, about 14 weeks are required to produce finished flowering plants in 11 cm pots.
   Plant height (from soil level to top of plant plane).—About 19 cm.
   Plant spread (width).—About 27 cm.
   Vigor.—Vigorous growth rate.
Foliage description:
   Leaf shape.—Single, dentate, cordate, palmate venation.
   Leaf length.—Basal: 25-45 mm Apical: 10-20 mm.
   Leaf width.—Basal: 20-40 mm Apical: 3-5 mm.
   Apex.—Acute.
   Base.—Cordate.
   Margin.—Broadly dentate.
   Texture.—Smooth, glabrous.
   Color.—Upper Surface, RHS N137A, Green Lower Surface, RHS N138A, Green.
Inflorescence description:
   Flower arrangement and shape.—Single, upright, campanulate flowers in racemes, flowers with small star shaped calyx.
   Natural flowering season.—Continuous throughout spring and summer. Season can be extended through long day treatments, no vernalization needed.
   Flower longevity.—Longevity of individual flowers is highly dependent on temperature and light conditions. Flowers persistent.
   Inflorescence size.—Length: 8-10 cm Diameter: about 3 cm.
   Flowers.—Length: About 5-10 mm Diameter: About 20-25 mm.
   Petal lobes.—Shape: Arrow-shaped Length: 8-10 mm Base width: 5 mm.
   Petals.—Overall Shape: Arrow-shaped. Length: 8-10 mm Width: 7 mm. Color: Upper surface: RHS 91A, violet-blue; bottom of bell N155D white Under surface: RHS 91B violet-blue; bottom of bell N155D white Base: Fused Margin: Entire.
   Buds.—Length: Up to 7 mm Diameter: Up to 4 mm Shape: Oblong, ridged Color: RHS 91A, violet-blue.
   Sepals.—Arrangement: Shiny, glabrous, free. Quantity per flower: 5.
   Length.—8 mm.
   Width.—2 mm.
   Overall shape.—Arrow-shaped.
   Apex.—Acute.
   Base.—Fused.
   Margin.—Entire.
   Color (mature and immature).—Upper and lower surfaces: Green, RHS 138A.
   Peduncle.—Strength: Average Length: About 3-10 mm Diameter: About 1 mm Color: RHS 138A, Green.
Reproductive organs:
   Pistil.—Quantity: 1 Length: about 8 mm.
   Stigma.—Shape: Tripartite Color: RHS 91A, violet-blue.
   Anther.—Shape: Fused, curling after shedding. Length: About 1 mm Color: RHS 158B, Yellow-White.
   Style.—Length: About 7 mm Color: Base RHS N155D, white; Apex RHS 91A, violet-blue.
   Ovary.—Color: RHS 150C, Yellow-green.
Weather tolerance: Plants of the new Campanula have exhibited good tolerance to drought, rain and wind, with low temperature resistance to −20° C.

I claim:

1. A new and distinct cultivar of Campanula plant named ‘PKMPOS01’, as illustrated and described herein.
